(19) United States
(12) Patent Application Publication
(10) Pub. No.: US 2013/0089887 A1
(43) Pub. Date: Apr. 11, 2013

(54) Method for Rapid Detection and Identification of micro-colonies using impregnated porous material

(71) Applicant: Sergey Gazenko, (US)

(72) Inventor: Sergey Gazenko, (US)

(21) Appl. No.: 13/269,798

(22) Filed: Oct. 10, 2011

(51) Int. Cl. C12Q 1/04; C12M 1/34

(52) U.S. Cl. 435/34; 435/288.7

(57) ABSTRACT

This invention describes method for rapid detection and identification of colonies and micro-colonies of microorganisms growing on the surface of certain nutrient agar under certain porous material(s) impregnated by chromogenic and/or flurogenic substrates and supportive substances. Growth in the space between nutrient agar and impregnated porous material allows limiting the negative influence of unfavorable substrates and substances and allows to reveal color or fluorescence spots at early stage of micro-colony growth.

### BACKGROUND OF THE INVENTION

The present invention relates generally to the field of microbiology and in particular to the field of rapid detection, identification, and enumeration of microorganisms colonies and micro-colonies.

Modern medicine, food and biotechnological industries, environmental control and military and civilian defense all have big need for rapid, sensitive, simple and cost-effective methods for microbiological analysis.

Detection and identification of microorganisms performs currently by different methods such as classical biochemical analysis, chromatography of fatty acids, enzyme-linked immune-sorbent assay (ELISA), mass-spectrometry, Fourier Transform Infra-Red (FTIR) spectroscopy, immuno-analyses like lateral flow devices, Polymerase Chain Reaction (PCR), Flow Cytometry and use of chromogenic or fluorogenic substrates dissolved in nutrient media for specific staining of target microorganisms. Some of these methods are rapid but not simple or/and cost-effective (PCR, Flow Cytometry, FTIR spectroscopy, mass-spectrometry), other need long preliminary growth and/or complicated analytical procedures (classical biochemical analysis, chromatography, immuno-analyses, nutrient agars with dissolved chromogens).

Current invention is most closely related to the group of methods using chromogenic and fluorogenic substrates, biochemical dyes and indicators for specific or general staining of microorganisms during or after certain growth period. These methods are CHROMagars™ and their numerous derivatives (U.S. Pat. Nos. 5,194,374; 5,846,761; 6,548,268; 7,198,907; 7,632,657), 3M™ Petrifilm™ plates (U.S. Pat. Nos. 4,565,783; 5,089,413; 5,232,838), Permeable Membrane method (US Patent application No. 20100190204). Common to all these methods is that the detection and/or identification is very simple and done without removing colonies or micro-colonies from the surface of nutrient media. CHROMagars™ and 3M™ Petrifilm™ plates allow very simple identification of colonies after regular growth time period by specific coloration of target colonies. Drawbacks of CHROMagars™ and 3M™ Petrifilm™ plates are: (1) long incubation period—24-48 or more hours, (2) necessity for big amount of chromogenic substrates and other substances (antibiotics, inhibitors) in order to create large concentration of chromogens in relatively big volume of nutrient agar (20-25 ml), (3) high cost of single plate ($10-15) due to necessity of big amount of costly substances, (4) short storage time of CHROMagars™ due to instability of chromogens and other substances in liquid environment of nutrient agar. Although permeable membrane method is rapid (⅓-¼ of regular time) it doesn't allow reliable identification. In this method micro-colonies are identified by their morphology but morphology of many species overlaps resulting in low reliability of analysis.

The above-described methods are able to use only chromogenic substrates. Fluorogenic substrates cannot be used as additives to nutrient agars due to huge background fluorescence of nutrient agars as a result of many different fluorescent components in agar.

Current invention overcomes these drawbacks and allows specific or non-specific detection and/or identification of microorganisms on nutrient agars much faster due to staining micro-colonies (early stage of colony forming). It is several times cost effective because smaller amounts of substances are needed. It opens new ways for use of chromogenic and fluorogenic substrates, and prolongs “shelf life” period of analytical substances because they are stored in a dry environment of a porous material.

### BRIEF DESCRIPTION OF THE INVENTION

A method for short time selective or non-selective growth of microorganisms and their detection and/or identification by staining with chromogenic and fluorogenic substrates together with other substances is provided.

Chromogenic and/or fluorogenic substrates are impregnated in porous material like polymeric membrane or paper. Porous material placed on nutrient agar containing target and non-target live cells that are ready to form micro-colonies. Thus, micro-colonies grow between nutrient agar and porous material filled by chromogenic or fluorogenic substrates.

Method of the main embodiment includes the following steps: (1) spreading of sample on the surface of nutrient agar, (2) placing porous material filled by certain chromogenic substrate(s) and other necessary substances (inhibitors, antibiotics, nutrient substances, etc.) on a surface of nutrient agar, (3) incubating ¼-½ of regular growth time and (4) analyzing the resulting colored spots created between nutrient agar and impregnated porous material which indicates target micro-colonies.

Method of another embodiment includes the following steps: (1) spreading of sample on the surface of nutrient agar, (2) growing micro-colonies within ⅛-½ of regular growth time and placing porous material impregnated by chromogenic substrate(s) and supportive chemicals on the surface of nutrient agar with already formed micro-colonies for a certain time, (3) analyzing the resulting color spots created between nutrient agar and impregnated porous material.

Method of another embodiment includes the following steps: (1) spreading of sample on the surface of nutrient agar, (2) placing black or non-fluorescent porous material filled with mixture of certain fluorogenic substrate(s) and supportive substances on the surface of nutrient agar, (3) incubating within ⅛-½ of regular growth time and (4) removing porous material from nutrient agar and analyzing the resulting fluorescent spots created on the surface of impregnated porous material at UV or other appropriate light.

According to another embodiment, porous material has one or more layer or certain material (protein, carbohydrate or other nature) on one side for protecting chromogenic and/or fluorogenic substrate from immediate reaction with enzymes of live cells. This layer is fermented by extracellular enzymes after which cells from micro-colonies come in contact with chromogenic or fluorogenic substrates.

According to another embodiment, more than one porous material is placed on nutrient agar. One porous material contains chromogenic/fluorogenic substrates for staining micro-colonies and the other contains certain critical substances for growth (special nutrient substance like one of carbohydrates, antibiotic, etc.). Thus micro-colonies will be formed and stained only if the critical substance meets the growth needs of the target.

According to another embodiment, porous material (one or more) contains not only chromogens/fluorogens and supportive substances like antibiotics but also certain amount of nutrient substances for maintaining growth of micro-colonies inside or outside of the surface of the porous material. These porous material(s) placed on pure agar with sample or just on a drop presumably containing target microorganisms. Color or fluorescent spots will appear inside porous material or on it's surface.

The use of chromogenic and fluorogenic substrates embedded in porous material (polymeric membrane of paper) for detection and identification purposes has following advantages: (1) the required amount of expensive substrates is several times less along with larger concentration in surface area, (2) ability to use not only highly soluble substrates but low soluble as well as insoluble, (3) ability to use larger concentrations of substrates that result in quicker detection of coloration, (4) cells growing under membrane or paper form flat colonies with much larger diameter that help their recognizing at early stage, (5) use of more than one porous material or combination of porous materials with soluble or insoluble layers allow creating many different new ways for rapid and simple detection and identification of many microorganisms.

### BRIEF DESCRIPTION OF THE FIGURES

FIG. 1 is a regular colony on the nutrient agar.

FIG. 2 is a micro-colony growing between porous material impregnated by artificial substrates and nutrient media.

FIG. 3 A is a single cell at start of incubation under two layers.

FIG. 3 B is a micro-colony that fermented one layer and reached the second layer.

FIG. 4 A and B demonstrate two versions of stained micro-colonies: A—by chromogenic substrates, B—by fluorogenic substrates.

### DETAILED DESCRIPTION OF THE INVENTION

The following terms and explanations are incorporated herein to define and elucidate the discussion of the invention below.

Nutrient agar: Nutrient agar is a growth medium used for growing colonies or micro-colonies within the context of the current invention. Nutrient agars can be a general medium for promoting bacterial growth such as Tryptic Soy Agar, a medium for fungi and yeast growth such as Saboraud Dextrose Agar, or a medium used for growth of special groups of microorganisms, such as Cetrimide Agar used for growth of Pseudomonas species, or MacConkey Agar used for the detection of Gram-negative enteric pathogens.

Micro-colony: A micro-colony is a small colony of cells appearing after 2-8 hours of incubation. The typical size of micro-colonies is 50-500 They are colorless and invisible under a regular light microscope. Therefore, micro-colonies typically need to be colored in order to become visible.

Porous material: A porous material is a layer of polymeric soluble or insoluble material able to imbibe dissolved or finely dispersed chromogenic or fluorogenic substrates and other substances. When solvent is evaporated, porous material is ready for use. Dried porous material with analytical substances placed on nutrient agar gets wet and starts releasing biologically active substances. Porous material can be cellulose acetate, nitrocellulose, and cellulose esters, polysulfone, polyether sulfone, polyamide, polyacrilonitrile, polyvinylidene fluoride, polyvinylchloride, cellulose (paper), agarose, gelatin, carrageenan or other natural or artificial polymers. Porosity allows gases (O2, CO2, air) to freely pass through the material and supports the growth of cells.

Analytical substances: Analytical substances are chromogenic and fluorogenic substrates (chromogens and fluorogens), biochemical indicators, inhibitors or accelerators of cells growth. Some examples of these substances are: 5-Bromo-4-chloro-3-indoxyl-N-acetyl-β-D-galactosaminide (blue precipitate with Glycosidases); 5-Bromo-4-chloro-3-indoxyl butyrate (blue precipitate with Esterases); 5-Bromo-4-chloro-3-indoxyl phosphate (blue precipitate with Phosphatases); 5-Iodo-3-indoxyl-β-D-galactopyranoside (purple precipitate with β-D-Galactosidase); 5-Bromo-6-chloro-3-indoxyl sulfate potassium salt (magenta with Sulfatase); 4-Methylumbelliferyl acetate (blue fluorescence with Esterases); L-Tryptophan 7-amido-4-methylcoumarin (blue fluorescence with L-Tryptopan peptidase); Tetrazolium Blue Chloride (blue precipitate with Dehydrogenases), Iodonitrotetrazolium Chloride (red precipitate with Dehydrogenases); Oxacillin sodium salt (inhibitor/antibiotic of Staphylococcus aureus growth); Galactose (carbohydrate supporting E. coli growth) and many other supported or inhibited growth or changing color or fluorescence organic and inorganic soluble and insoluble substances.

Detection and identification: The term “detection” is generally understood to mean the ability to reveal and count/enumerate any colonies or micro-colonies independent of their taxonomic classifications. “Identification” means the determination of genus and species of a given colony or micro-colony.

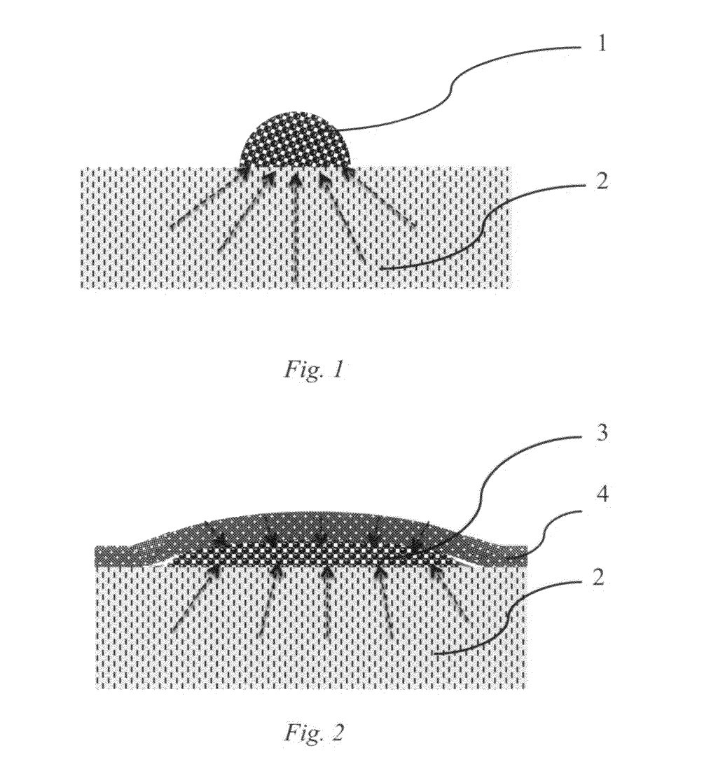

Regular growth of a colony as it takes place on CHROMagars™ is presented in FIG. 1. The colony 1 grows on nutrient agar 2 containing certain number and concentrations of chromogenic and other substances. Presence of staining substances and inhibitors in nutrient media doesn't support fast growth. Mixture of nutrients, staining and other substances reach the colony from one side—from the side of nutrient agar. Thus, nutrients supporting growth and substances for staining non-supporting growth are not separated: they reach the colony as a mixture. Arrows show the direction of flow of molecules from nutrient agar to the colony. Chromogens and inhibitors suppress the development of colony. Therefore it takes 24-48 hours or more to form colony of a regular size.

FIG. 2 presents main embodiment of invented method. Nutrient agar 2 and porous material 4 are separated and colony/micro-colony 3 is growing between them. The flow of nutrient substances to the colony and flow of chromogens with inhibitors are separated in space: the cells of colony 3 receives nutrients from nutrient agar and chromogens/fluorogens from the side of porous material. Majority of chromogenic/fluorogens substrates are not well soluble in water substances. Therefore, nutrients and chromogens/fluorogens are not mixed completely and both streams are significantly separated in space.

The volume of nutrient agar of CHROMagars™ in regular Petri plate is about 20 ml. It needs several tens of milligrams of expensive chromogenic substrates and inhibitors to create the required concentration for staining of colonies. Thus, high concentrations of analytical substances are required through the entire volume of agar in order to maintain just the surface layer. Concentration and amount of chromogens and inhibitors in porous material (invented method) is much lower than in CHROMagars™ or Petrifilm™ due to very small volume of thin porous material and it's proximity to the growing colony/micro-colony. This not only allows to increase the concentration of chromogens in porous material and, therefore increase intensity of colored spots on the surface of porous material, but also allows the lower part the of colony to grow faster due to lesser influence of chromogens/fluorogens and inhibitors.

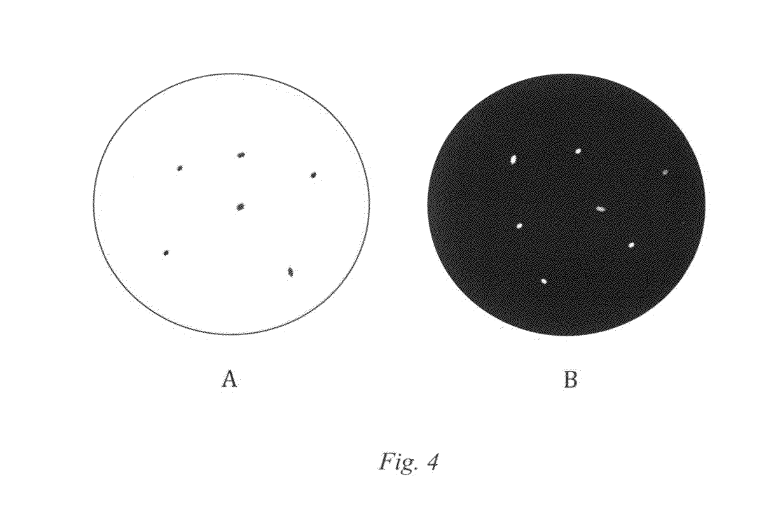

Color spots are clearly visible on the white surface of porous material even if they are small (FIG. 4 A): Fluorescent spots (FIG. 4 B) are well visible in UV or shorter wave light in case porous material is black or non-fluorescent.

Colony growing under relatively heavy porous material changes it shape from semi-spherical to more flat with larger area as can be seen in FIG. 1 (semi-spherical) and FIG. 2 (flat). Larger area of contact of colony/micro-colony to nutrient agar and to porous material is more favorable for better intake of substances by colony/micro-colony.

Larger size of colony/micro-colony, contrast of spots appeared on white surface of porous material, larger concentration of chromogens/fluorogens in porous material, separation of nutrient substances and chromogens/fluorogens allows the identification at much earlier stage—at the stage of micro-colony, within ¼-½ of regular incubation time.

Some chromogenic substrates are fermented slowly due to small concentration of corresponding enzymes or due to their slow turn-over. Others create color within a few minutes. Staining by “slow” chromogens (5-Bromo-4-chloro-3-indoxyl-N-acetyl-β-D-galactosaminide, 5-Iodo-3-indoxyl-β-D-galactopyranoside and other) require long (many hours) growth period under impregnated porous material in order to obtain visible spots. “Fast” chromogens/fluorogens (Fluoresceindiacetate, 4-Methylumbelliferyl phosphate and other) are fermented within minutes and do not need long incubation with cells. Therefore porous material 2 (FIG. 2) impregnated by “fast” substances can be mounted on nutrient agar on the already formed micro-colonies: after only 2-6 hours of incubation. Thus, color (FIG. 4 A) or fluorescent spots (FIG. 4 B) will appear within a few minutes. In the case of “fast” chromogens/fluorogens negative influence on cells growth is completely excluded and growth of cells and formation of micro-colony proceeds in most favorable conditions and significantly faster. Also, it is not necessary to remove porous material after micro-colony staining as colored spots can be seen clearly through nutrient agar.

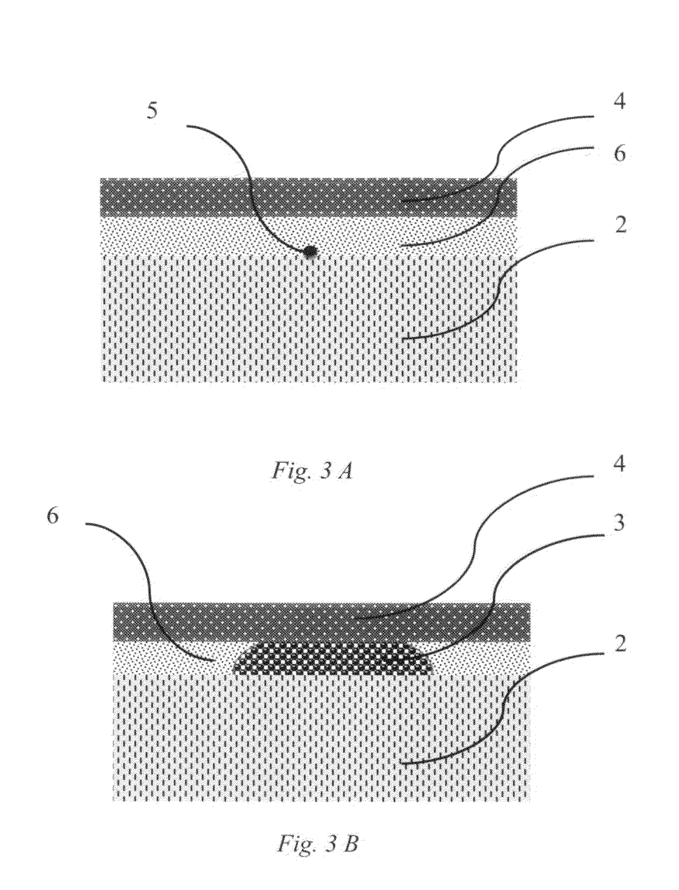

Number of porous materials on one nutrient agar can be more than one (FIGS. 3A and 3 B). The functions of additional porous materials can be different. They can be soluble or insoluble. Soluble porous material represented in FIG. 3A and FIG. 3B. According to this embodiment, formation of micro-colony (FIG. 3A) starts from one initial cell 5 settled under lower porous material 6. Porous material impregnated by chromogenic or fluorogenic substrates 4 settles above material 6. Thus free access of staining molecules for growing micro-colony is blocked. Material 6, if soluble or fermentable by target species will be dissolved after certain time and opens access to chromogenic/fluorogenic substances in second layer (FIG. 3B). Thus, it is known (Nan H. Albertson et al, 1990) that some Vibrio and Pseudomonas reveal exoproteases activity that enable the fermentation of proteins in surrounding space. They actively digest protein layer in case nutrient agar is low on proteins and/or amino acids. Example of such material 6 fermented by target micro-colonies can be thin layer of gelatin.

Porous material 6 can be insoluble (polymeric membrane or paper) but impregnated by soluble substances necessary for micro-colony growth. Example of such substances can be lactose fermented by lactose-positive E. coli growing on MacConkey nutrient agar but cannot be fermented by other bacteria. E. coli ferment lactose and produce acid. Acid changes color of pH indicator (Neutral red) to pink color. Thus colonies of lactose-positive organisms (mainly E. coli) become pink. Lactose-negative organisms remain colorless. According to current invention lactose and Neutral red are impregnated on two porous membranes: lactose on one (close to micro-colonies) and Neutral red on another. Concentration of lactose and Neutral red can be several times larger than in MacConkey agar but actual amount of both reagents are several times lower. Lactose-positive bacteria ferment lactose and produce acid that reveals pink spots where lactose-positive micro-colonies are localized. The intensity of color in this case will be much larger and it will appear earlier due to larger concentrations of both reagents. Thus lactose-positive bacteria will be revealed at early stage—at the stage of micro-colony formation. This example also shows that molecules participating in reaction can go not only from porous membrane to micro-colony but also in another direction: from micro-colony (acid) to upper porous material (Neutral red) by diffusion.

Detection of antibiotic-resistant microorganisms is another example of the advantage of current method. Methicillin resistant Staphylococcus aureus (MRSA) is a dangerous nosocomial infection. It can be detected by purple color of colonies after 24 hours of incubation if nutrient agar contains: 5-bromo-6-chloro-3-indoxyl phosphate (0.10 g/l of nutrient agar), 5-bromo-4-chloro-3-indoxyl glucoside (0.05 g/l), 5-bromo-4-chloro-3-indoxyl galactoside (0.05 g/l), 5-bromo-4-chloro-3-indoxyl glucuronide (0.05 g/l), Deferoxamine 0.050 g/l and Oxacillin (6 mg/ml) or cefoxitin (5 mg/l). (U.S. Pat. No. 7,632,657). Purple color of MRSA depends on 5-bromo-6-chloro-3-indoxyl phosphate and 5-bromo-4-chloro-3-indoxyl glucoside. Other colonies reveal other colors. According to the invented method chromogens and antibiotics mentioned above are not dissolved in nutrient agar before inoculation of sample but impregnated in porous material (nitrocellulose, cellulose acetate or cellulose) in smaller amounts but with concentration several times larger. Porous material is placed on nutrient agar after one hour of incubation, when micro-colonies have already formed couple of generations. After this, chromogens and antibiotics start slow dissolving which maintain big concentration of chromogens that are optimal for antibiotic only in upper layer of nutrient agar.

According to another embodiment one or more porous material contains not only chromogens/fluorogens and supportive substances like antibiotics but also certain amounts of nutrient substances supporting growth of colonies/micro-colonies inside or outside porous material. Those porous material(s) are placed on pure agar with sample or just on a drop presumably containing target microorganisms.

Porosity of Porous Material.

Porosity of porous material can be different depending on the design of analysis. Porosity is required for: (1) dissolving larger amount of reagents and keeping them inside the material after drying, (2) penetration of air through material to support growth of aerobic microorganisms, (3) for penetration of reagents from one layer to another if needed. If anaerobic or facultative anaerobic microorganisms are the subject of analysis, the upper material doesn't need to be porous. The size and number of pores in different layers can be different depending on the goals of the analysis. Thus, size and number of pores is important when speed of the transfer of molecules from one porous material to another must be faster or slower.

Impregnation of Porous Materials

Porous materials can be used not only with soluble substances but with insoluble substances as well. Substances (chromogenic/fluorogenic substrates, nutrient substances, antibiotics, etc.) of certain concentration are dissolved in appropriate solvent, which dissolves substances but not porous material. Solvent is removed by evaporation or by another method.

Chromogens/fluorogens and other substances can be insoluble or of low solubility in appropriate solvent. In this case porous material is treated by fine disperse, emulsified or colloidal mixture of reagent with solvent. Example of solvents: water, ethanol, methanol, acetone, dimethylsulfoxide (DMSO), dimethylformamide (DMF), acetonitrile, isopropanol and other.

Confirmation of Identification by Colony-Lifting Immuno-Assay and Dot-ELISA.

Taxonomical affiliation of micro-colonies detected or identified by invented method can be confirmed by already known immunological methods: Colony-Lifting Immunoassay and Dot-ELISA. This can be especially useful when the identification serotype of target microorganism is required. Questionable colored or fluorescent spots can be treated by antibody-enzyme conjugate, washed, and treated by chromogen for enzyme of conjugate by the above-mentioned known methods. Target spots will change color due to the presents of conjugate on the surface of cells. Not all porous materials can be used for this identification but only materials that strongly adhere cells/spots to its surface like Nitrocellulose or PTFE. Thus invented method can be helpful for confirmation of identification up to serotype if monoclonal antibody is used as a part of conjugate.

### EXAMPLE 1

### Rapid Identification of Micro-Colonies of Enterobacteriaceae

Nitrocellulose porous membrane disc is dipped in the mixture containing 5 mg of 5-Bromo-4-chloro-3-indolyl-β-D-galactopyranoside in one milliliter of PBS pH 9.0 for one minute. After this the membrane is then removed and dried on solid glass or plastic surface (polyethylene) surface. Sample presumably containing target cells is spread on the surface of nutrient agar. Dry porous membrane is placed on the nutrient agar and incubated at 38° C. Distinct blue spots appear after 8 hours of incubation. Each spot corresponds to target micro-colony of one of the species belonging to Enterobacteriaceae family. Rapid analysis of E. coli can be done by the same method but with use of 5-Bromo-4-chloro-3-indilyl-β-D-glucopyranoside.

### EXAMPLE 2

### Rapid Identification of MRSA

Two porous discs (nitrocellulose membrane) are prepared before analysis. First disc is dipped in mixture of chromogenic substrates: 5-bromo-6-chloro-3-indoxyl phosphate (0.2 mg/ml), 5-bromo-4-chloro-3-indoxyl glucoside (0.1 mg/ml), 5-bromo-4-chloro-3-indoxyl galactoside (0.1 mg/ml), 5-bromo-4-chloro-3-indoxyl glucuronide (0.1 mg/ml) in PBS pH 7.5 and dried. Second disc is dipped in mixture of Oxacillin (0.07 mg/ml) in PBS pH 7.5 and dried. Sample, presumably containing MRSA is spread on the surface of nutrient agar (TSA). First disc with chromogens is placed on the surface of agar. Plate is incubated for 1 hour at 38° C. Second disc with antibiotic is placed above the first disc and plate is incubated for 9 hours at the same temperature. Distinct purple color spots appear on the surface of the first nitrocellulose disc. They are identified as micro-colonies of MRSA. Micro-colonies of other species obtain other colors.

### EXAMPLE 3

### Rapid Identification of the Total Viable Organisms

Black nitrocellulose disc is dipped in a mixture of 4-Methylumbelliferyl acetate (0.1 mg/ml) and 4-Methylumbelliferyl phosphate (0.05 mg/ml) and PBS pH 8.0 and dried. Sample, presumably containing microorganisms is spread on the surface of nutrient agar (TSA) and incubated for 4 hours at 37° C. Dry disc is placed on the surface of nutrient agar for 10 minutes, removed and observed under UV light (λmax=360 nm). All micro-colonies fluoresce due to esterase and phosphatase activities present in all live cells and transform fluorogenic substrates to fluorescent substances.

I claim:

1. A method for rapid and simple detection and identification of colonies or micro-colonies of microorganisms comprising of growing sample presumably containing target microorganisms on nutrient agar under one or more porous materials impregnated by chromogenic and/or fluorogenic substrates and supportive substances.

2. A method of claim 1 wherein porous material contains substances in dry condition and release them during certain time after placing on the surface of nutrient agar and wetted.

3. A method of claim 1 wherein the period of incubation is shorter than regular incubation period by at least two times.

4. A method of claim 1 wherein chromogenic substrates are 3-Bromo-4-chloro-3-indoxyl-derivatives, 5-Bromo-6-chloro-3-indoxyl derivatives, 6-Chloro-3-indoxyl derivatives, N-Methylindoxyl derivatives, 3-indoxyl derivatives, 5-Iodo-3-indoxyl derivatives, Tetrazolium salts, pH indicators, biochemical dyes and other substances changing color on contact with live cells.

5. A method of claim 1 wherein fluorogenic substrates are Fluoresceine derivatives, 4-Methylumbelliferyl derivatives, 7-Amido-4-methylcoumarin derivatives, Rezorufine derivatives and other substances changing wavelength of fluorescence on contact with live cells.

6. A method of claim 1 wherein porous material cellulose acetate, nitrocellulose, cellulose esters, polysulfone, polyether sulfone, polytetrafluoroethylene, polyvinylidene fluoride, cellulose (paper), agarose, gelatin, carrageenan or other natural or artificial polymers are able to imbibe surrounding molecules and release them after wetting or allow contact of these molecules with growing or live cells.

7. A method of claim 1 wherein supportive substances are antibiotics, salts used in buffers for maintaining certain pH, carbohydrates, proteins and amino-acids, and other substances accelerating or inhibiting growth of colonies or micro-colonies.

8. A method of claim 1 wherein second porous material is fermentable or non-fermentable by cells.

9. A method of claim 1 wherein one porous material is non-penetrable for air.

10. A device for rapid detection and identification of colonies and/or micro-colonies comprising plate with nutrient agar and one or more porous materials impregnated by chromogenic and or fluorogenic substrates and other substances.
